SEQUENCE LISTING (31 sequences)

SEQ ID NO: 1 - protein, 5 aa, Homo sapiens
SYSMN

SEQ ID NO: 2 - protein, 17 aa, Homo sapiens
YISSSSSTIDYADSVKG

SEQ ID NO: 3 - protein, 9 aa, Homo sapiens
ESGWYLFDY

SEQ ID NO: 4 - protein, 11 aa, Homo sapiens
RASQGISSWLA

SEQ ID NO: 5 - protein, 7 aa, Homo sapiens
AASSLQS

SEQ ID NO: 6 - protein, 9 aa, Homo sapiens
QQYNSYPPT

SEQ ID NO: 7 - protein, 118 aa, Homo sapiens
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 8 - protein, 107 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 9 - protein, 444 aa, Homo sapiens
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 6 aa, Homo sapiens
STYWIS

SEQ ID NO: 12 - protein, 17 aa, Homo sapiens
KIYPGDSYTNYSPSFQG

SEQ ID NO: 13 - protein, 8 aa, Homo sapiens
RGYGIFDY

SEQ ID NO: 14 - protein, 11 aa, Homo sapiens
SGDNIGDQYAH

SEQ ID NO: 15 - protein, 7 aa, Homo sapiens
QDKNRPS

SEQ ID NO: 16 - protein, 11 aa, Homo sapiens
ATYTGFGSLAV

SEQ ID NO: 17 - protein, 116 aa, Homo sapiens
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 18 - protein, 108 aa, Homo sapiens
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 19 - protein, 442 aa, Homo sapiens
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 214 aa, Homo sapiens
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 22 - protein, 5 aa, Homo sapiens
DYAMH

SEQ ID NO: 23 - protein, 17 aa, Homo sapiens
GISWNSGSIGYADSVKG

SEQ ID NO: 24 - protein, 15 aa, Homo sapiens
DQSTADYYFYYGMDV

SEQ ID NO: 25 - protein, 11 aa, Homo sapiens
RASQSVSSYLA

SEQ ID NO: 26 - protein, 7 aa, Homo sapiens
DASNRAT

SEQ ID NO: 27 - protein, 8 aa, Homo sapiens
QQRSNWPT

SEQ ID NO: 28 - protein, 124 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSS

SEQ ID NO: 29 - protein, 106 aa, Homo sapiens
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 30 - protein, 450 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 213 aa, Homo sapiens
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0031765 A1
(43) Pub. Date: Jan. 31, 2019

(54) COMBINATION OF AN OX40 AGONIST AND A 4-1BB AGONIST MONOCLONAL ANTIBODY FOR TREATING CANCER

(71) Applicant: PFIZER INC., NEW YORK, NY (US)

(72) Inventors: HUA LONG, SAN CARLOS, CA (US); ARON DAVID THALL, SAN DIEGO, CA (US)

(73) Assignee: PFIZER INC., NEW YORK, NY (US)

(21) Appl. No.: 16/069,712

(22) PCT Filed: Jan. 17, 2017
(86) PCT No.: PCT/IB2017/050244

§ 371 Date: Jul. 12, 2018

Related U.S. Application Data
(60) Provisional application No. 62/286,616, filed on Jan. 25, 2016.

(51) Int. Cl. C07K 16/28; A61P 35/00

(52) U.S. Cl. CPC C07K 16/2878; A61P 35/00; A61K 2039/545; C07K 2317/565; C07K 2317/56; C07K 2317/75

(57) ABSTRACT

The present disclosure describes combination therapies comprising an agonist of OX40 and an agonist of 4-1BB, and the use of the combination therapies for the treatment of cancer.

### FIELD

The present invention relates to combination therapies useful for the treatment of cancer. In particular, the invention relates to a combination therapy which comprises an agonist of an OX40 protein and an agonist of a 4-1BB protein.

### BACKGROUND

Enhancing anti-tumor T cell function represents a powerful and novel approach for cancer treatment. Crucial components involved with generating an effective anti-tumor T cell response include enhancing CD4+ helper T cell activity to promote the generation of anti-tumor cytolytic T cells, and providing survival signals for memory and effector T cells.

The OX40 receptor (OX40) (also known as CD134, TNFRSF4, ACT-4, ACT35, and TXGP1L) is a member of the TNF receptor superfamily. OX40 is found to be expressed on activated T-cells. High numbers of OX40+ T cells have been demonstrated within tumors (tumor infiltrating lymphocytes) and in the draining lymph nodes of cancer patients (Weinberg, A. et al. J. Immunol. 164: 2160-69, 2000; Petty, J. et al. Am. J. Surg. 183: 512-518, 2002). It was shown in tumor models in mice that engagement of the OX40 in vivo during tumor priming significantly delayed and prevented the appearance of tumors as compared to control treated mice (Weinberg et al., 2000). Therefore, it has been contemplated to enhance the immune response of a mammal to an antigen by engaging OX40 through the use of an OX40 agonist (WO 99/42585; Weinberg et al., 2000).

4-1BB (CD137 and TNFRSF9), which was first identified as an inducible costimulatory receptor expressed on activated T cells, is a membrane spanning glycoprotein of the Tumor Necrosis Factor (TNF) receptor superfamily. Current understanding of 4-1BB indicates that expression is generally activation dependent and encompasses a broad subset of immune cells including activated NK and NKT cells; regulatory T cells; dendritic cells (DC) including follicular DC; stimulated mast cells, differentiating myeloid cells, monocytes, neutrophils, eosinophils (Wang C, et al. Immunol Rev. 229(1):192-215, 2009), and activated B cells (Zhang X, et al. J Immunol. 184(2):787-795, 2010). 4-1BB expression has also been demonstrated on tumor vasculature (Broil K, et al. Am J Clin Pathol. 115(4):543-549, 2001; Seaman S, et al. Cancer Cell 11(6):539-554, 2007) and atherosclerotic endothelium (21) (Olofsson P S, et al. Circulation (117(10):1292-1301, 2008). The ligand that stimulates 4-1BB (4-1BBL) is expressed on activated antigen-presenting cells (APCs), myeloid progenitor cells and hematopoeitic stem cells.

Interaction of 4-1BB on activated normal human B cells with its ligand at the time of B cell receptor engagement stimulates proliferation and enhances survival (Zhang X, et al. J Immunol. 184(2):787-795, 2010). The potential impact of 4-1BB engagement in B cell lymphoma has been investigated in two published studies. Evaluation of several types of human primary NHL samples indicated that 4-1BB was expressed predominantly on infiltrating T cells rather than the lymphoma cells (Houot R, et al. Blood 114(16):3431-3438, 2009). The addition of 4-1BB agonists to in vitro cultures of B lymphoma cells with rituximab and NK cells resulted in increased lymphoma killing (Kohrt H E, et al. Blood 117(8):2423-2432, 2011). In addition, B cell immunophenotyping was performed in two experiments using PF-05082566 in cynomolgus monkeys with doses from 0.001-100 mg/kg; in these experiments peripheral blood B cell numbers were either unchanged or decreased.

4-1BB is undetectable on the surface of naive T cells but expression increases upon activation. Upon 4-1BB activation, TRAF 1 and TRAF 2, which are pro-survival members of the TNFR-associated factor (TRAF) family, are recruited to the 4-1BB cytoplasmic tail, resulting in downstream activation of NFkB and the Mitogen Activated Protein (MAP) Kinase cascade including Erk, Jnk, and p38 MAP kinases. NFkB activation leads to upregulation of Bfl-1 and Bcl-XL, pro-survival members of the Bcl-2 family. The pro-apoptotic protein Bim is downregulated in a TRAF1 and Erk dependent manner (Sabbagh L, et al. J Immunol. 180(12):8093-8101, 2008).

Reports have shown that 4-1BB agonist mAbs increase costimulatory molecule expression and markedly enhance cytolytic T lymphocyte responses, resulting in anti-tumor efficacy in various models. 4-1BB agonist mAbs have demonstrated efficacy in prophylactic and therapeutic settings and both monotherapy and combination therapy tumor models and have established durable anti-tumor protective T cell memory responses (Lynch D H. Immunol Rev. 222:277-286, 2008). 4-1BB agonists also inhibit autoimmune reactions in a variety of autoimmunity models (Vinay D S, et al. J Mol Med. 84(9):726-736, 2006).

There is a need for improved therapies for the treatment of cancers. Furthermore, there is a need for therapies having greater efficacy than existing therapies. Preferred combination therapies of the present invention show greater efficacy than treatment with either therapeutic agent alone.

### SUMMARY

The invention relates to therapeutic regimens for the treatment of cancer.

In one embodiment, the invention provides a method for treating a cancer in an individual comprising administering to the individual a combination therapy which comprises an OX40 agonist and a 4-1BB agonist.

In another embodiment, the invention provides a medicament comprising an OX40 agonist for use in combination with a 4-1BB agonist for treating a cancer.

In yet another embodiment, the invention provides a medicament comprising a 4-1BB agonist for use in combination with an OX40 agonist for treating a cancer.

Other embodiments provide for use of an OX40 agonist in the manufacture of medicament for treating a cancer in an individual when administered in combination with a 4-1BB agonist and use of a 4-1BB agonist in the manufacture of a medicament for treating a cancer in an individual when administered in combination with an OX40 agonist.

In a still further embodiment, the invention provides for use of an OX40 agonist and a 4-1BB agonist in the manufacture of medicaments for treating a cancer in an individual. In some embodiments, the medicaments comprise a kit, and the kit also comprises a package insert comprising instructions for using the OX40 agonist in combination with a 4-1BB agonist to treat a cancer in an individual.

In embodiments of the treatment methods, medicaments, compositions, kits and uses provided herein, the OX40 agonist binds to the extracellular domain of OX40 and is capable of agonizing OX40. In some embodiments of the above treatment methods, medicaments and uses, the OX40 agonist is a monoclonal antibody. In one embodiment, the OX40 agonist is an OX40 antibody which comprises a heavy chain and a light chain, and wherein the heavy and light chain variable regions comprise the amino acid sequences shown in SEQ ID NO: 7 and SEQ ID NO: 8, respectively.

In some embodiments, the OX40 agonist is a monoclonal antibody which comprises a heavy chain variable region amino acid sequence as set forth in SEQ ID NO: 7.

In some embodiments, the OX40 agonist is a monoclonal antibody which comprises a light chain variable region amino acid sequence as set forth in SEQ ID NO: 8.

In some embodiments, the OX40 agonist is a monoclonal antibody which comprises a heavy chain variable region amino acid sequence as set forth in SEQ ID NO: 7, and further comprises a light chain variable region amino acid sequence as set forth in SEQ ID NO: 8.

In some embodiments, the OX40 agonist is a monoclonal antibody which comprises a heavy chain amino acid sequence as set forth in SEQ ID NO: 9 and further comprises a light chain amino acid sequence as set forth in SEQ ID NO: 10, with the proviso that the C-terminal lysine residue of SEQ ID NO: 9 is optionally absent.

In embodiments of the treatment method, medicaments, kits, compositions and uses provided herein, the 4-1BB agonist binds to the extracellular domain of 4-1BB and is capable of agonizing 4-1BB. In some embodiments of the above treatment method, medicaments and uses, the 4-1BB agonist is a monoclonal antibody.

In one embodiment, the isolated 4-1BB antibody binds human 4-1BB at an epitope located within amino acid residues 115-156 of SEQ ID NO: 21. In some embodiments, the 4-1BB antibody comprises the H-CDR1 amino acid sequence of SEQ ID NO: 11, H-CDR2 amino acid sequence of SEQ ID NO: 12 and H-CDR3 amino acid sequence of SEQ ID NO: 13. In some embodiments, the 4-1BB agonist is a monoclonal antibody which comprises the L-CDR1 amino acid sequence of SEQ ID NO: 14, L-CDR2 amino acid sequence of SEQ ID NO: 15, and L-CDR3 amino acid sequence of SEQ ID NO: 16.

In some embodiments, the 4-1BB agonist is a monoclonal antibody which comprises a heavy chain variable region amino acid sequence as set forth in SEQ ID NO: 17.

In some embodiments, the 4-1BB agonist is a monoclonal antibody which comprises a light chain variable region amino acid sequence as set forth in SEQ ID NO: 18.

In some embodiments, the 4-1BB agonist is a monoclonal antibody which comprises a heavy chain variable region amino acid sequence as set forth in SEQ ID NO: 17, and further comprises a light chain variable region amino acid sequence as set forth in SEQ ID NO: 18.

In some embodiments, the 4-1BB agonist is a monoclonal antibody which comprises a heavy chain amino acid sequence as set forth in SEQ ID NO: 19 and further comprises a light chain amino acid sequence as set forth in SEQ ID NO: 20, with the proviso that the C-terminal lysine residue of SEQ ID NO: 19 is optionally absent.

In some embodiments of the treatment methods, medicaments, compositions, kits and uses of the invention, the individual is a human and the cancer is a solid tumor and in some embodiments, the solid tumor is bladder cancer, breast cancer, clear cell kidney cancer, colon cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC), hepatocellular cancer, or triple negative breast cancer. In some embodiments, the cancer is an advanced solid tumor malignancy.

In other embodiments of the treatment methods, medicaments, compositions, kits and uses of the invention, the individual is a human and the cancer is a heme malignancy. In some embodiments, the heme malignancy is non-Hodgkin's lymphoma (NHL). In some embodiments, the heme malignancy is diffuse large B-cell lymphoma (DLBCL), EBV-positive DLBCL, follicular lymphoma, acute lymphoblastic leukemia (ALL), acute myeloid leukemia (AML), chronic lymphocytic leukemia (CLL), chronic myeloid leukemia (CML), primary mediastinal large B-cell lymphoma, mantle cell lymphoma (MCL), small lymphocytic lymphoma (SLL), T-cell/histiocyte-rich large B-cell lymphoma, Hodgkin's lymphoma (HL), multiple myeloma (MM), myeloid cell leukemia-1 protein (Mcl-1), or myelodysplastic syndrome (MDS).

In embodiments, medicaments provided herein comprise a pharmaceutically acceptable excipient.

In embodiments, provided herein is a kit which comprises a first container, a second container and a package insert, wherein the first container comprises at least one, two, three, four, five or ten doses of a medicament comprising an OX40 agonist, the second container comprises at least one, two, three, four, five, or ten doses of a medicament comprising a 4-1BB agonist, and the package insert comprises instructions for treating an individual for cancer using the medicaments.

In embodiments, a kit provided herein comprises at least a container and a package insert, wherein the container comprises at least one, two, three, four, five, or ten doses of a medicament comprising an OX40 agonist and a 4-1BB agonist, and the package insert comprises instructions for treating an individual for cancer using the medicament.

In embodiments, in methods, medicaments, uses, compositions or kits provided herein, the individual is a human and the OX40 agonist is a monoclonal antibody which specifically binds to human OX40.

In embodiments, in methods, medicaments, uses, compositions or kits provided herein, the OX40 agonist is a monoclonal antibody which comprises the heavy chain and light chain variable regions of SEQ ID NO: 7 and SEQ ID NO: 8, respectively.

In embodiments, in methods, medicaments, uses, compositions or kits provided herein, the OX40 agonist is a monoclonal antibody comprising: (a) heavy chain CDRs of SEQ ID NOs: 1, 2, and 3 and light chain CDRs of SEQ ID NOs: 4, 5, and 6; or (b) heavy chain CDRs of SEQ ID NOs: 22, 23, and 24 and light chain CDRs of SEQ ID NOs: 25, 26, and 27.

In embodiments, in methods, medicaments, uses, compositions, or kits provided herein, the OX40 agonist is a monoclonal antibody comprising: (a) a heavy chain variable region comprising SEQ ID NO: 7 and a light chain variable region comprising SEQ ID NO: 8; or (b) a heavy chain variable region comprising SEQ ID NO: 28 and a light chain variable region comprising SEQ ID NO: 29.

In embodiments, in methods, medicaments, uses, compositions or kits provided herein, the OX40 agonist is a monoclonal antibody comprising: (a) a heavy chain comprising SEQ ID NO: 9 and a light chain comprising SEQ ID NO: 10; or (b) a heavy chain comprising SEQ ID NO: 30 and a light chain comprising SEQ ID NO: 31.

In embodiments, in methods, medicaments, uses, compositions or kits provided herein, the 4-1BB agonist is a monoclonal antibody comprising heavy chain CDRs of SEQ ID NOs: 11, 12, and 13 and light chain CDRs of SEQ ID NOs: 14, 15, and 16.

In embodiments, in methods, medicaments, uses, compositions or kits provided herein, the 4-1BB agonist is a monoclonal antibody comprising a heavy chain variable region comprising SEQ ID NO: 17 and a light chain variable region comprising SEQ ID NO: 18.

In embodiments, in methods, medicaments, uses, compositions or kits provided herein, the 4-1BB agonist is a monoclonal antibody comprising a heavy chain comprising SEQ ID NO: 19 and a light chain comprising SEQ ID NO: 20.

In embodiments, in methods, medicaments, uses, compositions or kits provided herein relating to a cancer, the cancer is a solid tumor.

In embodiments, in methods, medicaments, uses, compositions or kits provided herein relating to a cancer, the cancer is carcinoma, lymphoma, leukemia, blastoma, sarcoma, bladder cancer, breast cancer, gastric cancer, clear cell kidney cancer, cervical cancer, head/neck squamous cell carcinoma (HNSCC), lung squamous cell carcinoma, malignant melanoma, non-small-cell lung cancer (NSCLC), ovarian cancer, pancreatic cancer, prostate cancer, renal cell cancer (RCC), hepatocellular carcinoma, small-cell lung cancer (SCLC), triple negative breast cancer, non-Hodgkin's lymphoma (NHL), diffuse large B-cell lymphoma (DLBCL), EBV-positive DLBCL, follicular lymphoma, acute lymphoblastic leukemia (ALL), acute myeloid leukemia (AML), chronic lymphocytic leukemia (CLL), chronic myeloid leukemia (CML), primary mediastinal large B-cell lymphoma, mantle cell lymphoma (MCL), small lymphocytic lymphoma (SLL), T-cell/histiocyte-rich large B-cell lymphoma, Hodgkin's lymphoma (HL), multiple myeloma (MM), myeloid cell leukemia-1 protein (Mcl-1), myelodysplastic syndrome (MDS), myeloma, glioma, renal cancer, liver cancer, lymphoblastic leukemia, lymphocytic leukemia, colorectal cancer, endometrial cancer, kidney cancer, thyroid cancer, bone cancer, brain cancer, stomach cancer, hepatoma, head and neck cancer, hepatobiliary cancer, central nervous system cancers, esophageal cancer, merkel cell carcinoma, testicular cancer, skin cancer, small intestine cancer, biliary cancer, neuroendocrine tumors, mesothelioma, uterine cancer, vulvar cancer, penile cancer, anal cancer, choriocarcinoma, thymic cancer, and oral cancer.

In embodiments, in methods, medicaments, uses, compositions or kits provided herein relating to treating a cancer in an individual, the individual has not been previously treated for an advanced solid malignant tumor.

In embodiments, in methods, medicaments, uses, compositions or kits provided herein, the 4-1BB agonist is PF-05082566.

In embodiments, in methods, medicaments, uses, compositions or kits provided herein, the OX40 agonist is PF-04518600.

In embodiments, provided herein is a method for treating cancer in an individual comprising administering to the individual a combination therapy which comprises an OX40 agonist and a 4-1BB agonist, wherein the OX40 agonist is a monoclonal antibody which comprises a heavy chain and a light chain, wherein the heavy and light chains comprise SEQ ID NO: 9 and SEQ ID NO: 10, respectively; and wherein the 4-1BB agonist is a monoclonal antibody which comprises a heavy chain and a light chain, wherein the heavy and light chains comprise SEQ ID NO: 19 and SEQ ID NO: 20, respectively.

In embodiments of methods provided herein involving treating cancer in an individual comprising administering to the individual a combination therapy which comprises an OX40 agonist and a 4-1BB agonist, the OX40 agonist and the 4-1BB agonist are administered simultaneously or sequentially. Optionally, the OX40 agonist is administered at a separate time from the 4-1BB agonist.

In embodiments of methods provided herein involving treating cancer in an individual comprising administering to the individual a combination therapy which comprises an OX40 agonist and a 4-1BB agonist, the OX40 agonist is administered every two weeks and the 4-1BB agonist is administered every four weeks.

In embodiments of methods provided herein involving treating cancer in an individual comprising administering to the individual a combination therapy which comprises an OX40 agonist and a 4-1BB agonist, the OX40 agonist is administered every one, two, three, or four weeks and the 4-1BB agonist is administered every one, two, three, or four weeks.

In embodiments of methods provided herein involving treating cancer in an individual comprising administering to the individual a combination therapy which comprises an OX40 agonist and a 4-1BB agonist, the OX40 agonist is administered every two weeks at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg and 10 mg/kg and the 4-1BB agonist is administered every four weeks at a fixed dose per subject selected from the group consisting of 1, 5, 10, 20, 30, 40, 50, 60, 70, 80, 90, 100, 150, 200, and 500 mg.

In embodiments of methods provided herein involving treating cancer in an individual comprising administering to the individual a combination therapy which comprises an OX40 agonist and a 4-1BB agonist, the OX40 agonist is administered every one, two, three, or four weeks at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, and 20 mg/kg and the 4-1BB agonist is administered every one, two, three, or four weeks at: a) a fixed dose per subject selected from the group consisting of 1, 5, 10, 20, 30, 40, 50, 60, 70, 80, 90, 100, 150, 200, and 500 mg, or b) a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, and 20 mg/kg.

In embodiments of methods provided herein involving treating cancer in an individual comprising administering to the individual a combination therapy which comprises an OX40 agonist and a 4-1BB agonist, the OX40 agonist is administered about every one, two, three, four, five, or six weeks at: a) a fixed dose per subject selected from the group consisting of about 0.1, 0.5, 1, 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg.

In embodiments of methods provided herein involving treating cancer in an individual comprising administering to the individual a combination therapy which comprises an OX40 agonist and a 4-1BB agonist, the 4-1BB agonist is administered about every one, two, three, four, five, or six weeks at: a) a fixed dose per subject selected from the group consisting of about 0.1, 0.5, 1, 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg.

In embodiments of methods provided herein involving treating cancer in an individual comprising administering to the individual a combination therapy which comprises an OX40 agonist and a 4-1BB agonist, the OX40 agonist is administered about every one, two, three, four, five, or six weeks at: a) a fixed dose per subject selected from the group consisting of about 0.1, 0.5, 1, 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg, and the 4-1BB agonist is administered about every one, two, three, four, five, or six weeks at: a) a fixed dose per subject selected from the group consisting of about 0.1, 0.5, 1, 2, 4, 5, 6, 8, 10, 20, 30, 40, 50, 60, 70, 80, 90, 100, 150, 200, 300, 400, 500, 600, 700, 800, 900, or 1000 mg, or b) a dose selected from the group consisting of about 0.01 mg/kg, 0.03 mg/kg, 0.1 mg/kg, 0.3 mg/kg, 1 mg/kg, 1.5 mg/kg, 3 mg/kg, 5 mg/kg, 10 mg/kg, 15 mg/kg, 20 mg/kg and 25 mg/kg.

In embodiments, provided herein is a medicament comprising an OX40 agonist for use in combination with a 4-1BB agonist for treating cancer in an individual, wherein the OX40 agonist is an a monoclonal antibody which comprises a heavy chain and a light chain, wherein the heavy and light chains comprise SEQ ID NO: 9 and SEQ ID NO: 10, respectively; and wherein the 4-1BB agonist is a monoclonal antibody which comprises a heavy chain and a light chain, wherein the heavy and light chains comprise SEQ ID NO: 19 and SEQ ID NO: 20, respectively.

In embodiments, provided herein is a medicament comprising a 4-1BB agonist, for use in combination with an OX40 agonist, for treating a cancer in an individual, wherein the OX40 agonist is a monoclonal antibody which comprises a heavy chain and a light chain, wherein the heavy and light chains comprise SEQ ID NO: 9 and SEQ ID NO: 10, respectively; and wherein the 4-1BB agonist is a monoclonal antibody which comprises a heavy chain and a light chain, wherein the heavy and light chains comprise SEQ ID NO: 19 and SEQ ID NO: 20, respectively.

In embodiments, in methods, medicaments, uses, compositions, or kits provided herein, the OX40 agonist is formulated as a liquid medicament which comprises 10 mg/ml OX40 agonist, excipients, and a histidine buffer, pH 5.5.

In embodiments, in methods, medicaments, uses, compositions or kits provided herein, the 4-1BB agonist is formulated as a liquid medicament which comprises 10 mg/ml 4-1BB agonist, α,α-trehalose dehydrate, dihydrate, disodium ethylenediaminetetraacetic acid dehydrate, polysorbate 80, and a histidine buffer, pH 5.5.

In embodiments, provided herein is a kit which comprises a first container, a second container and a package insert, wherein the first container comprises at least one dose of a medicament comprising an OX40 agonist, the second container comprises at least one dose of a medicament comprising a 4-1BB agonist, and the package insert comprises instructions for treating an individual for cancer using the medicaments, wherein the OX40 agonist is a monoclonal antibody which comprises a heavy chain and a light chain, wherein the heavy and light chains comprise SEQ ID NO: 9 and SEQ ID NO: 10, respectively; and wherein the 4-1BB agonist is a monoclonal antibody which comprises a heavy chain and a light chain, wherein the heavy and light chains comprise SEQ ID NO: 19 and SEQ ID NO: 20, respectively.

In embodiments, provided herein is a composition comprising an OX40 agonist for use in the treatment of cancer, wherein the OX40 agonist is for separate, sequential or simultaneous use in a combination with a 4-1BB agonist, and wherein the OX40 agonist is a monoclonal antibody comprising a heavy chain and a light chain, wherein the heavy and light chains comprise SEQ ID NO: 9 and SEQ ID NO: 10, respectively; and wherein the anti-4-1BB agonist is a monoclonal antibody comprising a heavy chain and a light chain, wherein the heavy and light chains comprise SEQ ID NO: 19 and SEQ ID NO: 20, respectively.

In embodiments, provided herein is a composition comprising a 4-1BB agonist for use in the treatment of cancer, wherein the 4-1BB agonist is for separate, sequential or simultaneous use in a combination with an OX40 agonist, and wherein the OX40 agonist is a monoclonal antibody which comprises a heavy chain and a light chain, wherein the heavy and light chains comprise SEQ ID NO: 9 and SEQ ID NO: 10, respectively; and wherein the 4-1BB agonist is a monoclonal antibody comprising a heavy chain and a light chain, wherein the heavy and light chains comprise SEQ ID NO: 19 and SEQ ID NO: 20, respectively.

In embodiments, in methods, medicaments, uses, compositions or kits provided herein comprising an OX40 agonist, the OX40 agonist is a monoclonal antibody that specifically binds to OX40 and comprises: a heavy chain variable region (VH) comprising a VH complementarity determining region one (CDR1), VH CDR2, and VH CDR3 of the VH comprising the amino acid sequence shown in SEQ ID NO 7; and a light chain variable region (VL) comprising a VL CDR1, VL CDR2, and VL CDR3 of the VL comprising the amino acid sequence shown in SEQ ID NO: 8. In embodiments, the OX40 monoclonal antibody comprises the VH CDR1 comprising the amino acid sequence shown in SEQ ID NO: 1, the VH CDR2 comprising the amino acid sequence shown in SEQ ID NO: 2, the VH CDR3 comprising the amino acid sequence shown in SEQ ID NO: 3, the VL CDR1 comprising the amino acid sequence shown in SEQ ID NO: 4, the VL CDR2 comprising the amino acid sequence shown in SEQ ID NO: 5, and the VL CDR3 comprising the amino acid sequence shown in SEQ ID NO: 6. In embodiments, the OX40 monoclonal antibody comprises a heavy chain comprising the amino acid sequence shown in SEQ ID NO: 9 and a light chain comprising the amino acid sequence shown in SEQ ID NO: 10.

In embodiments, in methods, medicaments, uses, compositions or kits provided herein comprising a 4-1BB agonist, the 4-1BB agonist is a monoclonal antibody that specifically binds to 4-1BB and comprises: a VH comprising a VH CDR1, VH CDR2, and VH CDR3 of the VH comprising the amino acid sequence shown in SEQ ID NO 17; and a VL comprising a VL CDR1, VL CDR2, and VL CDR3 of the VL comprising the amino acid sequence shown in SEQ ID NO: 18. In embodiments, the 4-1BB monoclonal antibody comprises the VH CDR1 comprising the amino acid sequence shown in SEQ ID NO: 11, the VH CDR2 comprising the amino acid sequence shown in SEQ ID NO: 12, the VH CDR3 comprising the amino acid sequence shown in SEQ ID NO: 13, the VL CDR1 comprising the amino acid sequence shown in SEQ ID NO: 14, the VL CDR2 comprising the amino acid sequence shown in SEQ ID NO: 15, and the VL CDR3 comprising the amino acid sequence shown in SEQ ID NO: 16. In embodiments, the 4-1BB monoclonal antibody comprises a heavy chain comprising the amino acid sequence shown in SEQ ID NO: 19 and a light chain comprising the amino acid sequence shown in SEQ ID NO: 20.

In embodiments, provided herein is a kit which comprises a first container, a second container and a package insert, wherein the first container comprises at least one dose of a medicament comprising an OX40 agonist, the second container comprises at least one dose of a medicament comprising a 4-1BB agonist, and the package insert comprises instructions for treating an individual for cancer using the medicaments.

In embodiments, provided herein is a composition comprising an OX40 agonist for use in the treatment of cancer wherein the OX40 agonist is for separate, sequential or simultaneous use in a combination with a 4-1BB agonist. In embodiments, provided herein is a composition comprising a 4-1BB agonist for use in the treatment of cancer wherein the 4-1BB agonist is for separate, sequential or simultaneous use in a combination with an OX40 agonist.

In embodiments, provided herein is a composition comprising an OX40 agonist for use in the treatment of cancer and a 4-1BB agonist for use in the treatment of cancer wherein the OX40 agonist and the 4-1BB agonist are combined or co-formulated.

In embodiments, in methods, medicaments, uses, compositions or kits provided herein comprising an OX40 agonist and an 4-1BB agonist, one or both of the agonists are administered via an intravenous, intramuscular, or subcutaneous route.

### BRIEF DESCRIPTION OF THE DRAWINGS

FIG. 1 depicts a graph summarizing tumor volume in response to treatment.

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of an OX40 agonist or 4-1BB agonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., OX40 or 4-1BB). Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., OX40 receptor) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to an OX40 epitope is an antibody that binds this epitope with greater affinity, avidity, more readily, and/or with greater duration than it binds to other OX40 epitopes or non-OX40 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach, a combination of both approaches, or by any other CDR definition provided herein.

A “CDR” of a variable region are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. “Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer (NSCLC), head and neck squamous cell carcinoma (HNSCC), glioma, Hodgkin's lymphoma, non-Hodgkin's lymphoma, diffuse large B-cell lymphoma (DLBCL), follicular lymphoma, acute lymphoblastic leukemia (ALL), acute myeloid leukemia (AML), chronic lymphocytic leukemia (CLL), chronic myeloid leukemia (CML), primary mediastinal large B-cell lymphoma, mantle cell lymphoma (MCL), small lymphocytic lymphoma (SLL), T-cell/histiocyte-rich large B-cell lymphoma, multiple myeloma, myeloid cell leukemia-1 protein (Mcl-1), myelodysplastic syndrome (MDS), gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, gastric cancer, bone cancer, Ewing's sarcoma, cervical cancer, brain cancer, stomach cancer, bladder cancer, hepatoma, breast cancer, colon carcinoma, hepatocellular carcinoma (HCC), clear cell renal cell carcinoma (RCC), head and neck cancer, hepatobiliary cancer, central nervous system cancers, esophageal cancer, malignant pleural mesothelioma, systemic light chain amyloidosis, lymphoplasmacytic lymphoma, myelodysplastic syndromes, myeloproliferative neoplasms, neuroendocrine tumors, merkel cell carcinoma, testicular cancer, and skin cancer.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” refers to a chemical or biological substance that can cause death of cancer cells, or interfere with growth, division, repair, and/or function of cancer cells. Examples of chemotherapeutic agents include those that are disclosed in WO 2006/129163, and US 20060153808, the disclosures of which are incorporated herein by reference. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that inhibit expression of genes implicated in abnormal cell proliferation or tumor growth. Chemotherapeutic agents useful in the treatment methods of the present invention include cytostatic and/or cytotoxic agents.

The antibodies and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art.

The term “simultaneous administration” as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

“Chothia” as used herein means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original | |
|---|---|
| residue | Conservative substitution |
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an OX40 agonist that consists essentially of a recited amino acid sequence may also include one or more amino acids, including substitutions of one or more amino acid residues, which do not materially affect the properties of the binding compound.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al. , (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al. , (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“Isolated antibody” and “isolated antibody fragment” refer to the purification status and in such context mean the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Patient” or “subject” refers to any single subject for which therapy is desired or that is participating in a clinical trial, epidemiological study or used as a control, including humans and mammalian veterinary patients such as cattle, horses, dogs, and cats.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer one or more therapeutic agents (e.g. a combination therapy of an OX40 agonist and a 4-1BB agonist) to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR or OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student's t-test, the chi^{t}-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

As used herein, “treatment” is an approach for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, or shrinking or decreasing the size of tumor.

“Ameliorating” as used herein means a lessening or improvement of one or more symptoms as compared to not administering an OX40 agonist and a 4-1BB agonist. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

The term “pharmaceutically acceptable carrier” refers to any inactive substance that is suitable for use in a formulation for the delivery of a binding molecule. A carrier may be an antiadherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Advanced solid tumor malignancy” and “advanced solid tumor” are used interchangeably to refer to a tumor that has relapsed, progressed, metastasized after, locally advanced, and/or is refractory to, the initial or first line treatment.

Advanced solid tumors include, but are not limited to, metastatic tumors in bone, brain, breast, liver, lungs, lymph node, pancreas, prostate, and soft tissue (sarcoma).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

The term “OX40 antibody” as used herein means an antibody, as defined herein, capable of binding to human OX40 receptor.

The terms “OX40” and “OX40 receptor” are used interchangeably in the present application, and refer to any form of OX40 receptor, as well as variants, isoforms, and species homologs thereof that retain at least a part of the activity of OX40 receptor. Accordingly, a binding molecule, as defined and disclosed herein, may also bind OX40 from species other than human. In other cases, a binding molecule may be completely specific for the human OX40 and may not exhibit species or other types of cross-reactivity. Unless indicated differently, such as by specific reference to human OX40, 0X40 includes all mammalian species of native sequence OX40, e.g., human, canine, feline, equine and bovine. One exemplary human OX40 is a 277 amino acid protein (UniProt Accession No. P43489).

“OX40 agonist antibody” as used herein means, any antibody, as defined herein, which upon binding to OX40, (1) stimulates or activates OX40, (2) enhances, increases, promotes, induces, or prolongs an activity, function, or presence of OX40, or (3) enhances, increases, promotes, or induces the expression of OX40. 0X40 agonists useful in the any of the treatment method, medicaments and uses of the present invention include a monoclonal antibody (mAb) which specifically binds to OX40.

Examples of mAbs that bind to human OX40, and useful in the treatment method, medicaments and uses of the present invention, are described in, for example, U.S. Pat. No. 7,960,515, PCT Patent Application Publication Nos. WO2009079335, WO201302823, and WO2013119202, and U.S. Patent Application Publication No. 20150190506, each of which is incorporated by reference herein in its entirety. In some embodiments an anti-OX40 antibody useful in the treatment, method, medicaments and uses disclosed herein is a fully human agonist monoclonal antibody comprising a heavy chain variable region and a light chain variable region comprising the amino acid sequences shown in SEQ ID NO: 7 and SEQ ID NO: 8, respectively. In some embodiments an anti-OX40 antibody useful in the treatment, method, medicaments and uses disclosed herein is a fully human agonist monoclonal antibody comprising a heavy chain variable region and a light chain variable region comprising the amino acid sequences shown in SEQ ID NO: 28 and SEQ ID NO: 29, respectively. In some embodiments, the anti-OX40 antibody is a fully human IgG2 or IgG1 antibody.

Table 2 below provides exemplary anti-OX40 antibody sequences for use in the treatment methods, medicaments and uses of the present invention.

TABLE 2
EXEMPLARY ANTI-HUMAN OX40 MONOCLONAL ANTIBODY
SEQUENCES

| CDRH1 11D4 | SYSMN (SEQ ID NO: 1) |
|---|---|
| CDRH2 11D4 | YISSSSSTIDYADSVKG (SEQ ID NO: 2) |
| CDRH3 11D4 | ESGWYLFDY (SEQ ID NO: 3) |
| CDRL1 11D4 | RASQGISSWLA (SEQ ID NO: 4) |
| CDRL2 11D4 | AASSLQS (SEQ ID NO: 5) |
| CDRL3 11D4 | QQYNSYPPT (SEQ ID NO: 6) |
| Heavy chain VR | EVQLVESGGGLVQPGGSLRLSCAASGFTFSSYSMNWV |
| 11D4 | RQAPGKGLEWVSYISSSSSTIDYADSVKGRFTISRDNAK |
| | NSLYLQMNSLRDEDTAVYYCARESGWYLFDYWGQGTL |
| | VTVSS (SEQ ID NO: 7) |
| Light chain VR | DIQMTQSPSSLSASVGDRVTITCRASQGISSWLAWYQQ |
| 11D4 | KPEKAPKSLIYAASSLQSGVPSRFSGSGSGTDFTLTISS |
| | LQPEDFATYYCQQYNSYPPTFGGGTKVEIK (SEQ ID |
| | NO: 8) |
| Heavy chain | EVQLVESGGGLVQPGGSLRLSCAASGFTFSSYSMNWV |
| 11D4 | RQAPGKGLEWVSYISSSSSTIDYADSVKGRFTISRDNAK |
| | NSLYLQMNSLRDEDTAVYYCARESGWYLFDYWGQGTL |
| | VTVSSastkgpsvfplapcsrstsestaalgclvkdyfpepvtvswnsgalts |
| | gvhtfpavlqssglyslssvvtvpssnfgtqtytcnvdhkpsntkvdktverkcc |
| | vecppcpappvagpsvflfppkpkdtlmisrtpevtcvvvdvshedpevqfn |
| | wyvdgvevhnaktkpreeqfnstfrvvsvltvvhqdwlngkeykckvsnkglp |
| | apiektisktkgqprepqvytlppsreemtknqvsltclvkgfypsdiavewes |
| | ngqpennykttppmldsdgsfflyskltvdksrwqqgnvfscsvmhealhnh |
| | ytqkslslspgk (SEQ ID NO: 9) |
| Light chain 11D4 | DIQMTQSPSSLSASVGDRVTITCRASQGISSWLAWYQQ |
| | KPEKAPKSLIYAASSLQSGVPSRFSGSGSGTDFTLTISS |
| | LQPEDFATYYCQQYNSYPPTFGGGTKVEIKrtvaapsvfifpp |
| | sdeqlksgtasvvcllnnfypreakvqwkvdnalqsgnsqesvteqdskdst |
| | yslsstltlskadyekhkvyacevthqglsspvtksfnrgec (SEQ ID NO: |
| | 10) |
| CDRH1 18D8 | DYAMH (SEQ ID NO: 22) |
| CDRH2 18D8 | GISWNSGSIGYADSVKG (SEQ ID NO: 23) |
| CDRH3 18D8 | DQSTADYYFYYGMDV (SEQ ID NO: 24) |
| CDRL1 18D8 | RASQSVSSYLA (SEQ ID NO: 25) |
| CDRL2 18D8 | DASNRAT (SEQ ID NO: 26) |
| CDRL3 18D8 | QQRSNWPT (SEQ ID NO: 27) |
| Heavy chain VR | EVQLVESGGGLVQPGRSLRLSCAASGFTFDDYAMHWV |
| 18D8 | RQAPGKGLEWVSGISWNSGSIGYADSVKGRFTISRDNA |
| | KNSLYLQMNSLRAEDTALYYCAKDQSTADYYFYYGMD |
| | VWGQGTTVTVSS (SEQ ID NO: 28) |
| Light chain VR | EIVVTQSPATLSLSPGERATLSCRASQSVSSYLAWYQQ |
| 18D8 | KPGQAPRLLIYDASNRATGIPARFSGSGSGTDFTLTISSL |
| | EPEDFAVYYCQQRSNWPTFGQGTKVEIK (SEQ ID NO: |
| | 29) |
| Heavy chain | EVQLVESGGGLVQPGRSLRLSCAASGFTFDDYAMHWV |
| 18D8 | RQAPGKGLEWVSGISWNSGSIGYADSVKGRFTISRDNA |
| | KNSLYLQMNSLRAEDTALYYCAKDQSTADYYFYYGMD |
| | VWGQGTTVTVSSastkgpsvfplapcsrstsestaalgclvkdyfpepv |
| | tvswnsgaltsgvhtfpavlqssglyslssvvtvpssnfgtqtytcnvdhkpsnt |
| | kvdktverkccvecppcpappvagpsvflfppkpkdtlmisrtpevtcvvvdv |
| | shedpevqfnwyvdgvevhnaktkpreeqfnstfrvvsvltvvhqdwlngke |
| | ykckvsnkglpapiektisktkgqprepqvytlppsreemtknqvsltclvkgfy |
| | psdiavewesngqpennykttppmldsdgsfflyskltvdksrwqqgnvfsc |
| | svmhealhnhytqkslslspgk (SEQ ID NO: 30) |
| Light chain 18D8 | EIVVTQSPATLSLSPGERATLSCRASQSVSSYLAWYQQ |
| | KPGQAPRLLIYDASNRATGIPARFSGSGSGTDFTLTISSL |
| | EPEDFAVYYCQQRSNWPTFGQGTKVEIKrtvaapsvfifpps |
| | deqlksgtasvvcllnnfypreakvqwkvdnalqsgnsqesvteqdskdsty |
| | slsstltlskadyekhkvyacevthqglsspvtksfnrgec (SEQ ID NO: |
| | 31) |

The term “4-1BB antibody” as used herein means an antibody, as defined herein, capable of binding to human 4-1BB receptor.

The terms “4-1BB” and “4-1BB receptor” are used interchangeably in the present application, and refer to any form of 4-1BB receptor, as well as variants, isoforms, and species homologs thereof that retain at least a part of the activity of 4-1BB receptor. Accordingly, a binding molecule, as defined and disclosed herein, may also bind 4-1BB from species other than human. In other cases, a binding molecule may be completely specific for the human 4-1BB and may not exhibit species or other types of cross-reactivity. Unless indicated differently, such as by specific reference to human 4-1BB, 4-1BB includes all mammalian species of native sequence 4-1BB, e.g., human, canine, feline, equine and bovine. One exemplary human 4-1BB is a 255 amino acid protein (Accession No. NM_001561; NP_001552). One embodiment of a complete human 4-1BB amino acid sequence is provided in SEQ ID NO: 21.

4-1BB comprises a signal sequence (amino acid residues 1-17), followed by an extracellular domain (169 amino acids), a transmembrane region (27 amino acids), and an intracellular domain (42 amino acids) (Cheuk ATC et al. 2004 Cancer Gene Therapy 11: 215-226). The receptor is expressed on the cell surface in monomer and dimer forms and likely trimerizes with 4-1BB ligand to signal.

“4-1BB agonist” as used herein means, any chemical compound or biological molecule, as defined herein, which upon binding to 4-1BB, (1) stimulates or activates 4-1BB, (2) enhances, increases, promotes, induces, or prolongs an activity, function, or presence of 4-1BB, or (3) enhances, increases, promotes, or induces the expression of 4-1BB. 4-1BB agonists useful in the any of the treatment method, medicaments and uses of the present invention include a monoclonal antibody (mAb) which specifically binds to 4-1BB. Alternative names or synonyms for 4-1BB include CD137 and TNFRSF9. In any of the treatment methods, medicaments and uses of the present invention in which a human individual is being treated, the 4-1BB agonists increase a 4-1BB-mediated response. In some embodiments of the treatment methods, medicaments and uses of the present invention, 4-1BB agonists markedly enhance cytotoxic T-cell responses, resulting in anti-tumor activity in several models.

Examples of mAbs that bind to human 4-1BB, and are useful in the treatment methods, medicaments and uses of the present invention, are described in U.S. Pat. No. 8,337,850 and US 2013-0078240, each of which is incorporated by reference herein in its entirety. Specific anti-human 4-1BB mAbs useful as the 4-1BB agonist in the treatment method, medicaments and uses of the present invention include, for example, PF-05082566. PF-05082566 is a fully humanized IgG2 agonist monoclonal antibody targeting 4-1BB.

In some embodiments an anti-4-1BB antibody useful in the treatment, method, medicaments and uses disclosed herein is a fully humanized IgG2 agonist monoclonal antibody comprising a heavy chain variable region and a light chain variable region comprising the amino acid sequences shown in SEQ ID NO: 17 and SEQ ID NO: 18, respectively.

Table 3 below provides exemplary anti-4-1BB antibody sequences for use in the treatment methods, medicaments and uses of the present invention.

TABLE 3
EXEMPLARY ANTI-HUMAN 4-1BB MONOCLONAL ANTIBODY
SEQUENCES

| | |
|---|---|
| CDRH1 | STYWIS (SEQ ID NO: 11) |
| CDRH2 | KIYPGDSYTNYSPSFQG (SEQ ID NO: 12) |
| CDRH3 | RGYGIFDY (SEQ ID NO: 13) |
| CDRL1 | SGDNIGDQYAH (SEQ ID NO: 14) |
| CDRL2 | QDKNRPS (SEQ ID NO: 15) |
| CDRL3 | ATYTGFGSLAV (SEQ ID NO: 16) |
| Heavy chain VR | EVQLVQSGAEVKKPGESLRISCKGSGYSFSTYWISWVR |
| | QMPGKGLEWMGKIYPGDSYTNYSPSFQGQVTISADKSI |
| | STAYLQWSSLKASDTAMYYCARGYGIFDYWGQGTLVT |
| | VSS (SEQ ID NO: 17) |
| Light chain VR | SYELTQPPSVSVSPGQTASITCSGDNIGDQYAHWYQQK |
| | PGQSPVLVIYQDKNRPSGIPERFSGSNSGNTATLTISGT |
| | QAMDEADYYCATYTGFGSLAVFGGGTKLTVL (SEQ ID |
| | NO: 18) |
| Heavy chain | EVQLVQSGAEVKKPGESLRISCKGSGYSFSTYWISWVR |
| | QMPGKGLEWMGKIYPGDSYTNYSPSFQGQVTISADKSI |
| | STAYLQWSSLKASDTAMYYCARGYGIFDYWGQGTLVT |
| | VSSastkgpsvfplapcsrstsestaalgclvkdyfpepvtvswnsgaltsgv |
| | htfpavlqssglyslssvvtvpssnfgtqtytcnvdhkpsntkvdktverkccve |
| | cppcpappvagpsvflfppkpkdtlmisrtpevtcvvvdvshedpevqfnwy |
| | vdgvevhnaktkpreeqfnstfrvvsvltvvhqdwlngkeykckvsnkglpap |
| | iektisktkgqprepqvytlppsreemtknqvsltclvkgfypsdiavewesng |
| | qpennykttppmldsdgsfflyskltvdksrwqqgnvfscsvmhealhnhytq |
| | kslslspgk (SEQ ID NO: 19) |
| Light chain | SYELTQPPSVSVSPGQTASITCSGDNIGDQYAHWYQQK |
| | PGQSPVLVIYQDKNRPSGIPERFSGSNSGNTATLTISGT |
| | QAMDEADYYCATYTGFGSLAVFGGGTKLTVLgqpkaaps |
| | vtlfppsseelqankatlyclisdfypgavtvawkadsspvkagvetttpskqsn |
| | nkyaassylsltpeqwkshrsyscqvthegstvektvaptecs (SEQ ID |
| | NO: 20) |

The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antibody is selected from the group consisting of Fab, Fab′-SH, F(ab′)₂, scFv and Fv fragments.

In some embodiments of the treatment methods, medicaments and uses of the present invention, the 4-1BB agonist is a monoclonal antibody which comprises: (a) light chain CDR SEQ ID NOs: 14, 15, and 16 and heavy chain CDR SEQ ID NOs: 11, 12, and 13.

In some embodiments of the treatment methods, medicaments and uses of the present invention, the OX40 agonist is a monoclonal antibody which comprises: (a) light chain CDR SEQ ID NOs: 4, 5, and 6, and heavy chain CDR SEQ ID NOs: 1, 2, and 3.

In some embodiments of the treatment methods, medicaments and uses of the present invention, the 4-1BB agonist is a monoclonal antibody which specifically binds to human 4-1BB and comprises (a) a heavy chain variable region comprising SEQ ID NO: 17 or a variant thereof, and (b) a light chain variable region comprising an amino acid sequence comprising SEQ ID NO: 18 or a variant thereof.

A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In some embodiments of the treatment methods, medicaments and uses of the present invention, the OX40 agonist is a monoclonal antibody which specifically binds to human OX40 and comprises (a) a heavy chain variable region comprising SEQ ID NO: 7 or a variant thereof, and (b) a light chain variable region comprising an amino acid sequence selected from the group consisting of SEQ ID NO: 8 or a variant thereof.

In some embodiments of the treatment methods, medicaments and uses of the present invention, the 4-1BB agonist is a monoclonal antibody which specifically binds to human 4-1BB and comprises (a) a heavy chain amino acid sequence as set forth in SEQ ID NO: 19 and (b) a light chain amino acid sequence as set forth in SEQ ID NO: 20, with the proviso that the C-terminal lysine residue of SEQ ID NO: 19 is optionally absent.

In some embodiments of the treatment methods, medicaments and uses of the present invention, the OX40 agonist is a monoclonal antibody which specifically binds to human OX40 and comprises (a) a heavy chain amino acid sequence as set forth in SEQ ID NO: 9 and (b) a light chain amino acid sequence as set forth in SEQ ID NO: 10, with the proviso that the C-terminal lysine residue of SEQ ID NO: 9 is optionally absent.

In some embodiments of the treatment methods, medicaments and uses of the present invention, the OX40 agonist is PF-04518600. PF-04518600 is a fully human IgG2 monoclonal antibody (mAb) that functions as an agonist for the OX40 receptor.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

II. Methods, Uses, and Medicaments

In one aspect of the invention, the invention provides a method for treating a cancer in an individual comprising administering to the individual a combination therapy which comprises an OX40 agonist and a 4-1BB agonist.

The combination therapy may also comprise one or more additional therapeutic agents. The additional therapeutic agent may be, e.g., a chemotherapeutic, a biotherapeutic agent (including but not limited to antibodies to VEGF, VEGFR, EGFR, Her2/neu, other growth factor receptors, CD20, CD40, CD-40L, CTLA-4, PD-L1 and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

In some embodiments, an additional therapeutic agent in a combination therapy provided herein comprising an OX-40 agonist and a 4-1BB agonist may be, for example, an anti-CTLA4 antibody, an anti-PD-1 antibody, an anti-PD-L1 antibody, an anti-TIM3 antibody, an anti-LAG3 antibody, an anti-TIGIT antibody, an anti-HVEM antibody, an anti-BTLA antibody, an anti-CD40 antibody, an anti-CD47 antibody, an anti-CSF1R or CSF1 antibody, an anti-MARCO antibody, a CCR2 inhibitor, a cytokine based therapy [for example, IL-2 (or IL-2 variants), IL-7 (and IL-7 variants), IL-15 (and IL-15 variants), IL-12 (and IL-12 variants), IFNγ (or IFNγ variants), IFNα (or IFNα variants) IL-8 or anti IL-8 antibodies], an anti-CXCR4 antibody, an anti-VEGFR1 or VEGFR2 antibody, TNFα (or TNFα variants), an anti-TNFR1 or TNFR2 antibody, a kinase inhibitor, an ALK inhibitor, a MEK inhibitor, an IDO inhibitor, a GLS1 inhibitor, anti-CD3 bispecific antibody, a CART cell or T cell therapy targeted therapy such as PTK7-ADC, an anti-tumor antibody [for example, an anti-CD19 antibody, an anti-CD20 antibody, or an anti-Her2 antibody], an oncolytic virus, or a tumor vaccine.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (e.g., in the same medicament or at the same time), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

Dosage units may be expressed in, for example, mg (i.e. mg per subject), mg/kg (i.e. mg/kg of body weight) or mg/m². The mg/m² dosage units refer to the quantity in milligrams per square meter of body surface area.

In some instances, the OX40 agonist and the 4-1BB agonist are combined or co-formulated in a single dosage form.

Although the simultaneous administration of the OX40 agonist and the 4-1BB agonist may be maintained throughout a period of treatment or prevention, anti-cancer activity may also be achieved by subsequent administration of one compound in isolation (for example, OX40 agonist without the 4-1BB agonist, following combination treatment, or alternatively the 4-1BB agonist, without OX40 agonist), following combination treatment.

In some embodiments, the 4-1BB agonist is administered before administration of the OX40 agonist, while in other embodiments, the 4-1BB agonist is administered after administration of the OX40 agonist.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan. In some embodiments, a combination therapy of the invention is used to treat an advanced stage tumor having dimensions of at least about 200 mm³, 300 mm³, 400 mm³, 500 mm³, 750 mm³, or up to 1000 mm³.

In one embodiment, the dosage regimen is tailored to the particular patient's conditions, response and associate treatments, in a manner which is conventional for any therapy, and may need to be adjusted in response to changes in conditions and/or in light of other clinical conditions.

In some embodiments, selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion, or by doses at intervals of, e.g., daily, every other day, three times per week, or one time each week, two weeks, three weeks, monthly, bimonthly, etc. A total weekly dose may be, for example, at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. Doses of thereapeutic agents provided herein may be provided to subjects, for example, on a per-mass basis (e.g. mg/kg) or on a fixed dose basis (e.g. mg/subject).

In some embodiments that employ an anti-human OX40 mAb as the OX40 agonist in the combination therapy, the dosing regimen will comprise administering the anti-human OX40 mAb at a dose of 0.01, 0.1, 0.3, 1, 1.5, 2, 3, 5, 6, 8, 10, 15, 20, 25, 50, 75, or 100 mg/kg at intervals of about 7 days (±2 days) or about 14 days (±2 days) or about 21 days (±2 days) or about 30 days (±2 days) throughout the course of treatment. In some embodiments, the dosing regimen will comprise administering the anti-human OX40 mAb at a dose of between about 0.01 mg/kg to about 25 mg/kg, at intervals of about 7 days (±2 days) or about 14 days (±2 days) or about 21 days (±2 days) or about 30 days (±2 days) throughout the course of treatment. In some embodiments, the dosing regimen will comprise administering the OX40 agonist at a fixed dose of 1, 5, 10, 15, 20, 25, 30, 35, 40, 45, 50, 60, 70, 80, 90, 100, 150, 200, 250, 300, 350, 400, 450, 500, 600, 700, 800, 900, or 1000 mg per subject at intervals of about 7 days (±2 days) or about 14 days (±2 days) or about 21 days (±2 days) or about 30 days (±2 days) throughout the course of treatment. In some embodiments, the dosing regimen will comprise administering the OX40 agonist at a fixed dose of between about 1 and 500 mg per subject at intervals of about 7 days (±2 days) or about 14 days (±2 days) or about 21 days (±2 days) or about 30 days (±2 days) throughout the course of treatment. In some embodiments, the dosing regimen will comprise administering the OX40 agonist at a fixed dose of between about 6 and 600 mg per subject at intervals of about 7 days (±2 days) or about 14 days (±2 days) or about 21 days (±2 days) or about 30 days (±2 days) throughout the course of treatment.

In other embodiments that employ an anti-human OX40 mAb as the OX40 agonist in the combination therapy, the dosing regimen will comprise administering the anti-human OX40 mAb at a dose of from about 0.005 mg/kg to about 10 mg/kg, from about 0.01 mg/kg to about 25 mg/kg, from about 0.1 mg/kg to about 50 mg/kg or from about 1 mg/kg to about 100 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the OX40 agonists described herein. In embodiments, the OX40 agonist is administered as a liquid medicament by IV infusion over a time period of about 30, 60, or 90 minutes. In embodiments, the OX40 agonist is administered as a liquid medicament which comprises 1, 5, 10, 20, 30, 40, 50, 60, 70, 80, 90, 100, 150, or 200 mg/mL OX40 agonist in an aqueous solution compounded in histidine buffer with excipients at pH 5.5. In embodiments, the OX40 agonist is supplied in sterilized 10 mL Type 1 clear glass vials with 20 mm serum stoppers and 20 mm aluminum flip-off seals, with a nominal fill volume of 10 mL.

In certain embodiments, the OX40 agonist in the combination therapy is administered intravenously at a dose selected from the group consisting of: 0.01 mg/kg Q2W (Q2W =one dose every two weeks), 0.1 mg/kg Q2W, 0.3 mg/kg Q2W, 1 mg/kg Q2W, 1.5 mg/kg Q2W, 2 mg/kg Q2W, 3 mg/kg Q2W, 5 mg/kg Q2W, 10 mg/kg Q2W, 0.01 mg/kg Q3W (Q3W =one dose every three weeks), 0.1 mg/kg Q3W, 0.3 mg/kg Q3W, 1 mg/kg Q3W, 1.5 mg/kg Q3W, 2 mg/kg Q3W, 3 mg/kg Q3W, 5 mg/kg Q3W, 10 mg/kg Q3W, 0.01 mg/kg Q4W (Q4W =one dose every four weeks), 0.1 mg/kg Q4W, 0.3 mg/kg Q4W, 1 mg/kg Q4W, 1.5 mg/kg Q4W, 2 mg/kg Q4W, 3 mg/kg Q4W, 5 mg/kg Q4W, and 10 mg/kg Q4W.

In certain embodiments, the OX40 agonist in the combination therapy comprises an anti-OX40 monoclonal antibody comprising a heavy chain variable region and a light chain variable region comprising the amino acid sequences shown in SEQ ID NO: 7 and SEQ ID NO: 8, respectively is administered intravenously at a dose selected from the group consisting of: 0.01 mg/kg Q2W, 0.1 mg/kg Q2W, 0.3 mg/kg Q2W, 1 mg/kg Q2W, 1.5 mg/kg Q2W, 2 mg/kg Q2W, 3 mg/kg Q2W, 5 mg/kg Q2W, 10 mg/kg Q2W, 0.01 mg/kg Q3W, 0.1 mg/kg Q3W, 0.3 mg/kg Q3W, 1 mg/kg Q3W, 1.5 mg/kg Q3W, 2 mg/kg Q3W, 3 mg/kg Q3W, 5 mg/kg Q3W, 10 mg/kg Q3W, 0.01 mg/kg Q4W, 0.1 mg/kg Q4W, 0.3 mg/kg Q4W, 1 mg/kg Q4W, 1.5 mg/kg Q4W, 2 mg/kg Q4W, 3 mg/kg Q4W, 5 mg/kg Q4W, and 10 mg/kg Q4W.

In certain embodiments, the OX40 agonist in the combination therapy comprises an anti-OX40 monoclonal antibody comprising a heavy chain and a light chain comprising the amino acid sequences shown in SEQ ID NO: 9 and SEQ ID NO: 10, respectively is administered intravenously at a dose selected from the group consisting of: 0.01 mg/kg Q2W, 0.1 mg/kg Q2W, 0.3 mg/kg Q2W, 1 mg/kg Q2W, 1.5 mg/kg Q2W, 2 mg/kg Q2W, 3 mg/kg Q2W, 5 mg/kg Q2W, 10 mg/kg Q2W, 0.01 mg/kg Q3W (Q3W =one dose every three weeks), 0.1 mg/kg Q3W, 0.3 mg/kg Q3W, 1 mg/kg Q3W, 1.5 mg/kg Q3W, 2 mg/kg Q3W, 3 mg/kg Q3W, 5 mg/kg Q3W, 10 mg/kg Q3W, 0.01 mg/kg Q4W (Q4W =one dose every four weeks), 0.1 mg/kg Q4W, 0.3 mg/kg Q4W, 1 mg/kg Q4W, 1.5 mg/kg Q4W, 2 mg/kg Q4W, 3 mg/kg Q4W, 5 mg/kg Q4W, and 10 mg/kg Q4W.

In some embodiments, the dosing regimen will comprise administering the 4-1BB agonist at a dose of 0.01, 0.1, 0.5, 1, 2, 3, 5, 6, 8, 10, 15, 20, 25, 50, 75, or 100 mg/kg at intervals of about 7 days (±2 days) or about 14 days (±2 days) or about 21 days (±2 days) or about 30 days (±2 days) throughout the course of treatment. In some embodiments, the dosing regimen will comprise administering the 4-1BB agonist at a dose of between about 0.01 mg/kg to about 25 mg/kg, at intervals of about 7 days (±2 days) or about 14 days (±2 days) or about 21 days (±2 days) or about 30 days (±2 days) throughout the course of treatment. In some embodiments, the dosing regimen will comprise administering the 4-1BB agonist at a fixed dose of 1, 5, 10, 15, 20, 25, 30, 35, 40, 45, 50, 60, 70, 80, 90, 100, 150, 200, 250, 300, 350, 400, 450, 500, 600, 700, 800, 900, or 1000 mg per subject at intervals of about 7 days (±2 days) or about 14 days (±2 days) or about 21 days (±2 days) or about 30 days (±2 days) throughout the course of treatment. In some embodiments, the dosing regimen will comprise administering the 4-1BB agonist at a fixed dose of between about 1 and 500 mg per subject at intervals of about 7 days (±2 days) or about 14 days (±2 days) or about 21 days (±2 days) or about 30 days (±2 days) throughout the course of treatment. In some embodiments, the dosing regimen will comprise administering the 4-1BB agonist at a fixed dose of between about 6 and 600 mg per subject at intervals of about 7 days (±2 days) or about 14 days (±2 days) or about 21 days (±2 days) or about 30 days (±2 days) throughout the course of treatment.

In other embodiments, the dosing regimen will comprise administering the 4-1BB agonist at a dose of from about 0.005 mg/kg to about 10 mg/kg, from about 0.01 mg/kg to about 25 mg/kg, from about 0.1 mg/kg to about 50 mg/kg, or from about 1 mg/kg to about 100 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In another embodiment of the invention, the 4-1BB agonist in the combination therapy is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W, 1 mg/kg Q4W, 2 mg/kg Q4W, 3 mg/kg Q4W, 5 mg/kg Q4W, and 10 mg/kg Q4W.

In another embodiment of the invention, the 4-1BB agonist in the combination therapy comprises an anti-4-1BB monoclonal antibody comprising a heavy chain variable region and a light chain variable region comprising the amino acid sequences shown in SEQ ID NO: 17 and SEQ ID NO: 18, respectively, and is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W, 1 mg/kg Q4W, 2 mg/kg Q4W, 3 mg/kg Q4W, 5 mg/kg Q4W, and 10 mg/kg Q4W.

In another embodiment of the invention, the 4-1BB agonist in the combination therapy comprises an anti-4-1BB monoclonal antibody comprising a heavy chain and a light chain comprising the amino acid sequences shown in SEQ ID NO: 19 and SEQ ID NO: 20, respectively, and is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W, 1 mg/kg Q4W, 2 mg/kg Q4W, 3 mg/kg Q4W, 5 mg/kg Q4W, and 10 mg/kg Q4W.

In another embodiment of the invention, the 4-1BB agonist in the combination therapy comprises an anti-4-1BB monoclonal antibody comprising a heavy chain variable region and a light chain variable region comprising the amino acid sequences shown in SEQ ID NO: 17 and SEQ ID NO: 18, respectively, and is administered in a liquid medicament at a dose selected from the group consisting of 1, 5, 10, 20, 30, 40, 50, 60, 70, 80, 90, 100, 150, 200, 300, 400, 500, 600, 700, 800, 900, or 1000 mg per subject at a frequency of Q2W, Q3W, or Q4W.

In another embodiment of the invention, the 4-1BB agonist in the combination therapy comprises an anti-4-1BB monoclonal antibody comprising a heavy chain variable region and a light chain variable region comprising the amino acid sequences shown in SEQ ID NO: 19 and SEQ ID NO: 20, respectively, and is administered in a liquid medicament at a dose selected from the group consisting of 1, 5, 10, 20, 30, 40, 50, 60, 70, 80, 90, 100, 150, 200, 300, 400, 500, 600, 700, 800, 900, or 1000 mg per subject at a frequency of Q2W, Q3W, or Q4W.

In some embodiments, the 4-1BB agonist is administered as a liquid medicament, and the selected dose of the medicament is administered by IV infusion over a time period of about 30, 60, or 90 minutes.

The optimal dose for a particular OX40 agonist in combination with a particular 4-1BB agonist may be identified by dose escalation of one or both of these agents.

In an embodiment, a combination therapy provided herein may comprise administering to a subject an OX40 agonist at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg or 10 mg/kg at a frequency of Q2W, Q3W, or Q4W and a 4-1BB agonist at a fixed dose of 1, 5, 10, 20, 30, 40, 50, 60, 70, 80, 90, 100, 150, 200, 300, 400, 500, 600, 700, 800, 900, or 1000 mg per subject at a frequency of Q2W, Q3W, or Q4W.

In an embodiment, a combination therapy provided herein may comprise administering to a subject an OX40 agonist at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg or 10 mg/kg at a frequency of Q2W (one dose every two weeks) and a 4-1BB agonist at a fixed dose of 1, 5, 10, 20, 30, 40, 50, 60, 70, 80, 90, 100, 150, 200, 300, 400, 500, 600, 700, 800, 900, or 1000 mg per subject at a frequency of Q4W (one dose every four weeks).

In embodiments, in a combination therapy provided herein, on days in which a subject is to receive a dose of both OX40 agonist and 4-1BB agonist, the OX40 agonist and 4-1BB agonist are administered to the subject at time intervals separated by least 5, 10, 15, 30, or 60 minutes and no more than 360 minutes.

In an embodiment, an OX40 agonist is administered at a starting dose of 0.01 mg/kg, 0.03 mg/kg, 0.1 mg/kg, 0.3 mg/kg, 1 mg/kg, 1.5 mg/kg, 3 mg/kg, 5 mg/kg or 10 mg/kg Q2W and a 4-1BB agonist is administered Q4W at a starting fixed dose of 1, 5, 10, 20, 30, 40, 50, 60, 70, 80, 90, or 100 mg per subject.

In an embodiment, an OX40 agonist is administered at a starting dose of 2 mg/kg Q2W and a 4-1BB agonist is administered Q4W at a starting dose of 0.3 mg/kg, 0.6 mg/kg, 1.2 mg/kg, 2.4 mg/kg, or 5 mg/kg.

In another embodiment, an OX40 agonist is administered at a starting dose of 2 mg/kg Q3W and a 4-1BB agonist is administered Q3W at a starting dose of 0.3 mg/kg, 0.6 mg/kg, 1.2 mg/kg, 2.4 mg/kg, or 5 mg/kg.

In yet another embodiment, a 4-1BB agonist is administered at a starting dose of 0.6 mg/kg Q4W and an OX40 agonist is administered at a starting dose of 10 mg/kg Q2W, and if the starting dose combination is not tolerated by the patient, then the dose of an OX40 agonist is reduced to 2 mg/kg Q2W and/or the dose of 4-1BB agonist is reduced to 0.3 mg/kg Q4W.

In an embodiment, the dosage regimen is any combination of an OX40 agonist ata dose selected from the group consisting of 2 mg/kg Q2W and 10 mg/kg Q2W, and 4-1BB agonist at a dose selected from the group consisting of 1.2 mg/kg Q4W, 2.4 mg/kg Q4W and 5.0 mg/kg Q4W.

In embodiments, exemplary dosage regimens for a combination of OX40 agonist and 4-1BB agonist are provided in Table 4:

TABLE 4
Exemplary OX40 and 4-1BB dosage regimens

| OX40 agonist | and | 4-1BB agonist |
|---|---|---|
| 0.01 mg/kg Q2W | and | 10 mg Q4W |
| 0.01 mg/kg Q2W | and | 20 mg Q4W |
| 0.1 mg/kg Q2W | and | 10 mg Q4W |
| 0.1 mg/kg Q2W | and | 20 mg Q4W |
| 0.3 mg/kg Q2W | and | 20 mg Q4W |
| 0.3 mg/kg Q2W | and | 100 mg Q4W |
| 1 mg/kg Q2W | and | 100 mg Q4W |
| 3 mg/kg Q2W | and | 100 mg Q4W |

In some embodiments, dosage levels below the lower limit of the aforesaid range may be more than adequate, while in other cases still larger doses may be employed, as determined by those skilled in the art.

In some embodiments, a treatment cycle begins with the first day of combination treatment and last for 3 weeks or 4 weeks. On any day of a treatment cycle that the drugs are co-administered, in embodiments, the OX40 agonist infusion begins 30 minutes after completion of the infusion of the 4-1BB agonist. Alternatively, the OX40 agonist is administered by IV infusion after completion of the 4-1BB agonist infusion. In embodiments, the OX40 agonist and the 4-1BB agonist may be administered by simultaneous IV infusion.

In some embodiments, a combination therapy provided herein is administered for at least 12 weeks (three 4 week cycles or four 3 week cycles), more preferably at least 24 weeks, and even more preferably at least 2 to 4 weeks after the patient achieves a complete regression.

In some embodiments, the patient selected for treatment with the combination therapy of the invention has been diagnosed with an advanced solid malignant tumor. In embodiments, the patient has not received prior systemic therapy for the advanced tumor.

The present invention also provides a medicament which comprises an OX40 agonist as described above and a pharmaceutically acceptable excipient. When the OX40 agonist is a biotherapeutic agent, e.g., a mAb, the agonist may be produced in CHO cells using conventional cell culture and recovery/purification technologies.

In some embodiments, a medicament comprising an anti-OX40 antibody as the OX40 agonist may be provided as a liquid formulation or prepared by reconstituting a lyophilized powder with sterile water for injection prior to use. In some embodiments, a medicament comprising OX40 agonist is provided in a glass vial which contains about 100 mg of OX40 agonist.

The present invention also provides a medicament which comprises a 4-1BB agonist antibody and a pharmaceutically acceptable excipient. The 4-1BB agonist antibody may be prepared as described in, for example, U.S. Pat. No. 8,337,850 or US20130078240.

In some embodiments, the 4-1BB agonist antibody may be formulated at a concentration of 10 mg/mL to allow intravenous (IV). The commercial formulation may contain L-histidine buffer with α,α-trehalose dihydrate, disodium ethylenediaminetetraacetic acid dihydrate and polysorbate 80 at pH 5.5.

The OX40 and 4-1BB medicaments described herein may be provided as a kit which comprises a first container and a second container and a package insert. The first container contains at least one dose of a medicament comprising an OX40 agonist, the second container contains at least one dose of a medicament comprising a 4-1BB agonist, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some embodiments of the kit, the OX40 agonist is an anti-OX40 antibody. In some embodiments of the kit, the 4-1BB agonist is an anti-4-1BB antibody.

In some embodiments of a kit provided herein, a container of the kit contains both OX40 agonist and 4-1BB agonist in the same container. In some embodiments of a kit provided herein, the OX40 agonist and 4-1BB agonist are provided in separate containers.

Incorporated by reference herein for all purposes is the content of U.S. Provisional Patent Application No. 62/286,616 (filed Jan. 25, 2016).

These and other aspects of the invention, including the exemplary specific embodiments listed below, will be apparent from the teachings contained herein.

III. General Methods

Standard methods in molecular biology are described Sambrook, Fritsch and

Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John

Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Colo. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, N.Y.; Harlow and Lane (1988) Antibodies A Laboratory Manual, Cold Spring Harbor Laboratory Press, Cold Spring Harbor, N.Y., pp. 139-243; Carpenter, et al. (2000)J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### IV. EXAMPLES

### Example 1

### Combination Activity of Anti-OX40 (OX86 mlpG1) and Anti-4-1-BB Monoclonal Antibodies in CT26 Colon Carcinoma Mouse Model

The potential combinatorial effect of an anti-OX40 antibody mlgG1 and an anti-4-1BB mlgG1 antibody was evaluated in vivo in the murine CT26 colon carcinoma syngeneic tumor model.

For this study, an agonist anti-mouse OX40 antibody in the mouse IgG1 framework (mouse equivalent of human IgG2 in terms of mouse fragment crystallizable gamma receptor [FcγR] binding) was generated from parental clone OX86. The anti-4-1BB antibody used in this study was a mouse IgG1 agonist anti-mouse 4-1BB antibody.

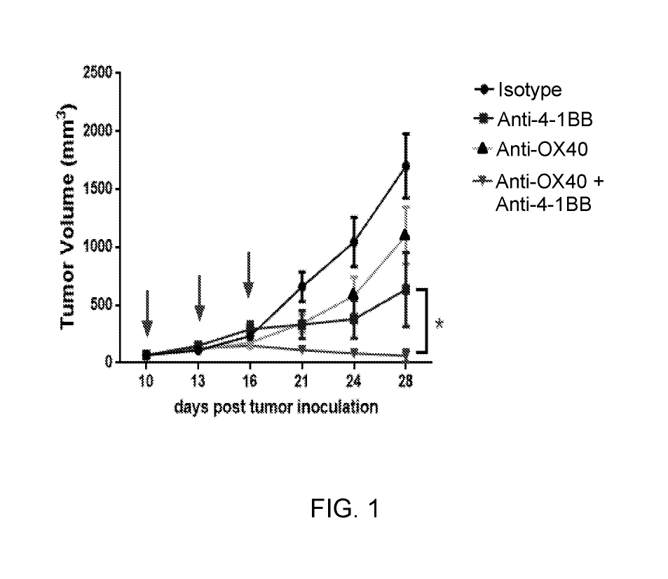

CT26 tumor cells (0.1×10⁶) were inoculated subcutaneously in female Balb/C mice. On Day 10 after tumor cell inoculation, average tumor size reached 67 mm³, and mice were randomized into treatment groups (10 mice/group). Female Balb/c mice were treated intraperitoneally on Days 10, 13, and 16 after tumor cell inoculation with 0.1 mg/kg of anti-4-1BB antibody, 0.03 mg/kg of anti-OX40 antibody, the combination of 0.1 mg/kg of anti-4-1BB antibody and 0.03 mg/kg of anti-OX40 antibody, or an isotype control antibody. Tumor growth inhibition was measured until Day 28. Tumor measurements were conducted in a blinded fashion twice per week throughout the study. Tumor growth inhibition on Day 28 was calculated by normalizing the difference between the treatment groups and the isotype control group. The results are summarized in FIG. 1 [X-axis is days post-tumor inoculation; Y-axis is tumor volume, mm³; down arrows indicate antibody treatment days; and symbols indicate antibody treatment (square: anti-4-1BB monotherapy; triangle: anti- OX40 monotherapy; inverted triangle: combination anti-4-1BB and anti-OX40 therapy; circle: isotype control)]. Anti-4-1BB antibody monotherapy and anti-OX40 monotherapy led to tumor growth inhibition of 63.3% and 35.3%, respectively on Day 28. The combination treatment resulted in 96.4% inhibition on Day 28 after tumor inoculation compared to the isotype control-treated animals (FIG. 1, Table 5). The combination was significantly better than either single antibody treatment alone by two-way ANOVA analysis with a p value of at least <0.05. This study was carried out through Day 41. While all animals in the isotype control-treated group developed tumors, the anti-OX40 monotherapy group and the anti-4-1BB monotherapy group had 1 out of 10 and 7 out of 10 animals, respectively, that were tumor free on Day 41 after tumor inoculation. In the combination group, 9 out of 10 animals were tumor free at Day 41 (Table 5). Furthermore, the combination of both the anti-OX40 antibodies and the anti-4-1BB antibodies were well-tolerated in this model as no decreases in body weight and no obvious toxicities were noted.

These data demonstrate the combination treatment with anti-OX40 antibody and anti-4-1BB antibody results in greater tumor growth inhibition than treatment with either antibody alone.

TABLE 5
Tumor Growth Inhibition of CT26 Murine Colon Cancer Syngeneic
Model After Combination Treatment OX86 mlgG1 and Anti-4-1BB

| | | | Tumor | |
|---|---|---|---|---|
| | | | Growth | Tumor |
| Treatment | Mean ± SEM | | Inhibition | Free |
| Groups | mm³ | N | (Day 28) | (Day 41) |
| Isotype control | 1701 ± 279 | 10 | 0% | 0/10 |
| 0.2 mg/kg | | | | |
| Anti-OX40 | 1101 ± 247, * | 10 | 35.3% | 1/10 |
| 0.03 mg/kg | | | | |
| Anti-4-1BB 0.1 mg/kg | 625 ± 320, **** | 10 | 63.3% | 7/10 |
| Anti-OX40 | 61 ± 55, ****, † | 10 | 96.4% | 9/10 |
| 0.03 mg/kg + | | | | |
| Anti-4-1BB 0.1 mg/kg | | | | |

N = Number;
SEM = Standard error of the mean;
* = Statistical significance comparing to control group, p < 0.05;
**** = p < 0.0001;
† = Statistical significance compared to anti-4-1BB, p < 0.05.

### Example 2

### Combination Activity of Anti-OX40 (OX86 mIciG1) and Anti-4-1-BB Monoclonal Antibodies in B16-F10 Melanoma Mouse Model

The combination of surrogate agonist anti-OX40 and anti-4-1BB antibodies described in Example 1 were also studied in the B16-F10 melanoma syngeneic model, a less immunogenic model with less T-cell infiltration in the tumor. C57BL/6 mice were inoculated with B16-F10 cells and then on Days 11, 14, 17 and 21 after tumor cell inoculation they were treated with isotype control antibody, a combination of 5 mg/kg anti-OX40 antibody and 1 mg/kg anti-4-1BB antibody, or each of the single agents. Results showed that the combination of anti-OX40 antibody and anti-4-1BB antibody did not inhibit the growth of established tumors in this aggressive model, consistent with published data (Gray et al., Eur J Immunol. 38(9): 2499-511, 2008).

In a separate experiment, B16-F10 (0.3×106) cells were injected into C57BI6 mice. On Days 11, 14, 18 after tumor cell inoculation they were treated with isotype control antibody, a combination of 3 mg/kg anti-OX40 antibody and 1 mg/kg anti-4-1BB antibody, or each of the single agents (4 mice per group). Tumor and spleen were harvested on Day 19 to investigate changes in T-cell phenotypes in the tumor and spleen. Cells were dissociated and stained with CD4, CD8, CD45, Ki67 antibodies, and viability dye. Data were acquired by flow cytometry and statistics were analyzed by one-way ANOVA. Statistical analyses were done comparing to isotype control group. In the tumor, while anti-OX40 monotherapy treatment slightly increased CD4 T-cell infiltration, the combination of anti-OX40 and anti-4-1BB antibodies significantly increased the CD4 T-cell percentage from 8.07±1.15% in the isotype control-treated animals to 19.78±3.70% (p<0.05). Similarly, CD8 tumor infiltration was increased from 8.62±1.18% in the isotype control group to 27.55±1.78% with the combination treatment (p<0.01). In the spleen, although overall CD4 and CD8 T-cell percentages didn't change significantly, cell proliferation was significantly increased with 63.98±6.36% of CD4 T cells expressing proliferation marker Ki67 in the combination group as compared to 27.53±2.31% in the isotype control group (p<0.0001). In addition, proliferating CD8 T cells in the spleen increased from 28.30±1.49% in isotype control group to 67.10±5.23% in the combination group (p<0.0001) (Table 6).

These data demonstrate the combination treatment with anti-OX40 antibody and anti-4-1BB antibody results in greater T-cell proliferation and tumor infiltration than treatment with either antibody alone.

TABLE 6
Anti-OX40 and Anti-4-1BB Costimulation Increase T-Cell
Proliferation and Tumor Infiltration in the Murine B16-F10 Melanoma Model

| | | | | Anti-OX40 + |
|---|---|---|---|---|
| | Isotype Control | Anti-OX40 | Anti-4-1BB | Anti-4-1BB |
| Tumor CD4% | 8.07 ± 1.15 | 13.86 ± 2.15 | 11.39 ± 1.61 | 19.78 ± 3.70, * |
| Tumor CD8% | 8.62 ± 1.18 | 13.25 ± 0.82 | 22.25 ± 5.27, * | 27.55 ± 1.78, |
| | | | | ** |
| Spleen CD4 | 27.53 ± 2.31 | 45.45 ± 1.43, * | 38.10 ± 2.98 | 63.98 ± 6.36, |
| Ki67% | | | | **** |
| Spleen CD8 | 28.30 ± 1.49 | 36.70 ± 3.13 | 43.45 ± 3.22, * | 67.10 ± 5.23, |
| Ki67% | | | | **** |

* = Statistical significance comparing to isotype control antibody.
* = p < 0.05,
**, p < 0.01,
**** = p < 0.0001;
CD = Cluster of differentiation;
Ki67 = Kiel 67 protein

### Example 3

### Combination Treatment with OX40 Aqonist and 4-1BB Aqonist

This example illustrates a clinical trial study to evaluate one or more of safety, efficacy, anti-tumor activity, pharmacokinetics, pharmacodynamics, and biomarker modulation of an anti-OX40 antibody in combination with an anti-4-1BB antibody in patients with selected advanced or solid metastatic solid tumors.

One objective of the study is to assess safety and tolerability at increasing dose levels of an anti-OX40 antibody in combination with an anti-4-1BB antibody in patients with selected advanced or metastatic solid tumors and to estimate MTD (Maximum Tolerated Dose) of the combination. The combination therapy dose escalation phase, will enroll approximately 53 patients. Sequential dose levels of an anti-OX40 antibody (0.1, 0.3, 1.0 and 3 mg/kg) combined with 20 mg or 100 mg of an anti-4-1BB antibody in adult patients with NSCLC, HNSCC, melanoma, bladder, gastric or cervical cancer who are unresponsive to currently available therapies or for whom no standard therapy is available. The starting dose level will be 0.1 mg/kg of anti-OX40 antibody and 20 mg of anti-4-1BB antibody, given no sooner than 30 minutes apart.

The anti-4-1BB antibody will be administered on Day 1 of every other cycle (every 28 days) as an intravenous (IV) infusion over 60 minutes (+/−5 minutes). The anti-4-1BB antibody will be administrated intravenously using a fixed dose. The anti-OX40 antibody will be administered on Day 1 of each 14-day cycle as an intravenous (IV) infusion over 60 minutes (+/−5 minutes) on an outpatient basis.

The anti-OX40 antibody will be administered intravenously with adjustment for body weight at every cycle. On cycles whereby both the anti-OX40 antibody and the anti-4-1BB antibody are to be administered on the same day, the anti-OX40 antibody will be administered after, but no sooner than 30 minutes after completion of the anti-4-1BB antibody infusion in absence of infusion reaction and after post-anti-4-1BB antibody and pre-anti-OX40 antibody pharmacokinetic blood draws.

A cycle is defined as the time from Day 1 dose of anti-OX40 antibody to the next Day 1 dose. If there are no treatment delays, a cycle will be 14 days. Each patient may receive anti-OX40 antibody and anti-4-1BB antibody until disease progression, unacceptable toxicity, withdrawal of consent, or study termination.

Optionally, the starting dose of anti-OX40 antibody will be 0.01 mg/kg combined with 20 mg of anti-4-1BB antibody.

For dose escalation, an initial 2 to 4 patients may be enrolled initially into each dose level combination. The starting dose combination level will be 0.1 mg/kg anti-OX40 antibody combined with 20 mg of anti-4-1BB antibody. If no DLTs are observed, the next dose combination level will be 0.3 mg/kg anti-OX40 antibody combined with 20 mg of anti-4-1BB antibody. If no toxicity is observed, the dose of anti-OX40 antibody and/or anti-4-1BB antibody will continue to be increased, to combination levels of 0.3 mg/kg anti-OX40 antibody combined with 100 mg of anti-4-1BB antibody, 1 mg/kg anti-OX40 antibody combined with 100 mg of anti-4-1BB antibody, and 3 mg/kg anti-OX40 antibody combined with 100 mg of anti-4-1BB antibody. If toxicity is observed at the starting dose combination level, 0.1 mg/kg anti-OX40 antibody combined with 10 mg of anti-4-1BB antibody will be evaluated. Subsequent to the initial dose, if dose de-escalation is recommended after evaluation, intermediate dose levels between the previous dose combination and current dose combination may be studied. Using the observed data, 1 or more dose combination levels of anti-OX40 antibody and anti-4-1BB antibody with toxicity rate closest to, but not exceeding, the predefined target rate of 25% will be identified. If the starting dose is deemed not tolerable, the next dose combination level will be 0.1 mg/kg anti-OX40 antibody combined with 10 mg of anti-4-1BB antibody. Dose levels of 0.01 mg/kg anti-OX40 antibody combined with 10 mg of anti-4-1BB antibody or 0.01 mg/kg anti-OX40 antibody combined with 20 mg of anti-4-1BB antibody may also be provided.

When a dose combination level is deemed safe following a DLT observation period of 28 days or 2 cycles (of anti-OX40 antibody), escalation will occur to the next dose combination level. A staggered start will be employed for all dose combination levels; that is, the first patient for any dose combination level will be dosed, and observed for 48 hours before subsequent patients can be dosed. If no safety concerns arise during this 48 hour period, a second patient will be enrolled into the same dose combination level.

Peripheral pharmacodynamic assessments of any given dose combination level may be completed after the dose combination level is deemed safe, and escalation to the next dose combination level has already occurred. When peripheral monitoring indicates immune modulation in the first 2-4 patients, the dose level will be expanded to approximately 10 patients allowing better characterization of pharmacodynamic effects and reducing variability due to small sample size. To allow for better characterization of pharmacodynamic effects, these additional patients will undergo mandatory pre-treatment and on treatment biopsies. If no peripheral pharmacodynamic effects are observed for the first 2-4 patients in any dose combination level, the dose combination level will not be expanded.

The combination therapy dose expansion phase will further evaluate safety and anti-tumor activity of the combination into 2 arms: arm 1 will enroll HNSCC patients who have never been treated with anti-PD-L1 or anti-PD-1 mAb; arm 2 will enroll NSCLC patients who have 1) previously received prior anti-PD-L1 or anti-PD-1 mAb as most recent therapy, and 2) did not have progressive disease as best overall response on recent PD-L1/PD-1 therapy, and 3) who subsequently progressed, or are intolerant to this therapy. This portion of the study will initially enroll up to 20 patients in each arm, and all patients will undergo a mandatory pre- and on-treatment tumor biopsy. The dose level of anti-OX40 antibody and the dose level of anti-4-1BB antibody within the dose combination level will be selected on initial data from the combination therapy, and may include, for example, any of the combination dose levels described above.

The studies above may generate data relevant to one or more of safety, efficacy, anti-tumor activity, pharmacokinetics, pharmacodynamics, and biomarker modulation of a combination treatment of the anti-OX40 antibody in combination with the anti-4-1BB antibody in patients with selected advanced or solid metastatic solid tumors.

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for treating cancer in an individual comprising administering to the individual a combination therapy which comprises an OX40 agonist and a 4-1BB agonist.

2. The method of claim 1, wherein the OX40 agonist is a monoclonal antibody that specifically binds to OX40 and comprises:
a heavy chain variable region (VH) comprising a VH complementarity determining region one (CDR1), VH CDR2, and VH CDR3 of the VH comprising the amino acid sequence shown in SEQ ID NO: 7; and
a light chain variable region (VL) comprising a VL CDR1, VL CDR2, and VL CDR3 of the VL comprising the amino acid sequence shown in SEQ ID NO: 8.

3. The method of claim 1, wherein the 4-1BB agonist is a monoclonal antibody that specifically binds to 4-1BB and comprises:
a VH comprising a VH CDR1, VH CDR2, and VH CDR3 of the VH comprising the amino acid sequence shown in SEQ ID NO 17; and
a VL comprising a VL CDR1, VL CDR2, and VL CDR3 of the VL comprising the amino acid sequence shown in SEQ ID NO: 18.

4. The method of claim 1,
wherein the OX40 monoclonal antibody comprises the VH CDR1 comprising the amino acid sequence shown in SEQ ID NO: 1, the VH CDR2 comprising the amino acid sequence shown in SEQ ID NO: 2, the VH CDR3 comprising the amino acid sequence shown in SEQ ID NO: 3, the VL CDR1 comprising the amino acid sequence shown in SEQ ID NO: 4, the VL CDR2 comprising the amino acid sequence shown in SEQ ID NO: 5, and the VL CDR3 comprising the amino acid sequence shown in SEQ ID NO: 6; and
wherein the 4-1BB monoclonal antibody comprises the VH CDR1 comprising the amino acid sequence shown in SEQ ID NO: 11, the VH CDR2 comprising the amino acid sequence shown in SEQ ID NO: 12, the VH CDR3 comprising the amino acid sequence shown in SEQ ID NO: 13, the VL CDR1 comprising the amino acid sequence shown in SEQ ID NO: 14, the VL CDR2 comprising the amino acid sequence shown in SEQ ID NO: 15, and the VL CDR3 comprising the amino acid sequence shown in SEQ ID NO: 16.

5. The method of claim 1, wherein the OX40 monoclonal antibody comprises a heavy chain comprising the amino acid sequence shown in SEQ ID NO: 9 and a light chain comprising the amino acid sequence shown in SEQ ID NO: 10.

6. The method of claim 1, wherein the 4-1BB monoclonal antibody comprises a heavy chain comprising the amino acid sequence shown in SEQ ID NO: 19 and a light chain comprising the amino acid sequence shown in SEQ ID NO: 20.

7. (canceled)

8. The method of claim 1, wherein the OX40 agonist is administered at a separate time from the 4-1BB agonist.

9. The method of claim 1, wherein the OX40 agonist is administered every two weeks and the 4-1BB agonist is administered every four weeks.

10. The method of claim 1, wherein the OX40 agonist is administered every two weeks at a dose selected from the group consisting of 0.01 mg/kg, 0.03 mg/kg, 0.1 mg/kg, 0.3 mg/kg, 1 mg/kg, 1.5 mg/kg, 3 mg/kg, 5 mg/kg and 10 mg/kg and the 4-1BB agonist is administered every four weeks at a fixed dose per subject selected from the group consisting of 1, 5, 10, 20, 30, 40, 50, 60, 70, 80, 90, 100, 150, 200, and 500 mg.

11. The method of claim 1, wherein the cancer is a solid tumor.

12. The method of claim 1, wherein the cancer is melanoma, head/neck squamous cell carcinoma (HNSCC), hepatocellular carcinoma, renal cell carcinoma (RCC), bladder cancer, cervical cancer, gastric cancer, or non-small cell lung cancer (NSCLC).

13. A medicament comprising an OX40 agonist, for use in combination with a 4-1BB agonist, for treating cancer in an individual.

14. The medicament of claim 13,
wherein the OX40 agonist is a monoclonal antibody that specifically binds to OX40 and comprises:
a heavy chain variable region (VH) comprising a VH complementarity determining region one (CDR1), VH CDR2, and VH CDR3 of the VH comprising the amino acid sequence shown in SEQ ID NO 7; and
a light chain variable region (VL) comprising a VL CDR1, VL CDR2, and VL CDR3 of the VL comprising the amino acid sequence shown in SEQ ID NO: 8.

15. The medicament of claim 13,
wherein the 4-1BB agonist is a monoclonal antibody that specifically binds to 4-1BB and comprises:
a VH comprising a VH CDR1, VH CDR2, and VH CDR3 of the VH comprising the amino acid sequence shown in SEQ ID NO 17; and
a VL comprising a VL CDR1, VL CDR2, and VL CDR3 of the VL comprising the amino acid sequence shown in SEQ ID NO: 18.

16. The medicament of claim 13,
wherein the OX40 monoclonal antibody comprises the VH CDR1 comprising the amino acid sequence shown in SEQ ID NO: 1, the VH CDR2 comprising the amino acid sequence shown in SEQ ID NO: 2, the VH CDR3 comprising the amino acid sequence shown in SEQ ID NO: 3, the VL CDR1 comprising the amino acid sequence shown in SEQ ID NO: 4, the VL CDR2 comprising the amino acid sequence shown in SEQ ID NO: 5, and the VL CDR3 comprising the amino acid sequence shown in SEQ ID NO: 6; and
wherein the 4-1BB monoclonal antibody comprises the VH CDR1 comprising the amino acid sequence shown in SEQ ID NO: 11, the VH CDR2 comprising the amino acid sequence shown in SEQ ID NO: 12, the VH CDR3 comprising the amino acid sequence shown in SEQ ID NO: 13, the VL CDR1 comprising the amino acid sequence shown in SEQ ID NO: 14, the VL CDR2 comprising the amino acid sequence shown in SEQ ID NO: 15, and the VL CDR3 comprising the amino acid sequence shown in SEQ ID NO: 16.

17. The medicament of claim 13, wherein the OX40 monoclonal antibody comprises a heavy chain comprising the amino acid sequence shown in SEQ ID NO: 9 and a light chain comprising the amino acid sequence shown in SEQ ID NO: 10.

18. The medicament of claim 13, wherein the 4-1BB monoclonal antibody comprises a heavy chain comprising the amino acid sequence shown in SEQ ID NO: 19 and a light chain comprising the amino acid sequence shown in SEQ ID NO: 20.

19-24. (canceled)

25. A kit which comprises a first container, a second container and a package insert, wherein the first container comprises at least one dose of a medicament comprising an OX40 agonist, the second container comprises at least one dose of a medicament comprising a 4-1BB agonist, and the package insert comprises instructions for treating an individual for cancer using the medicaments.

26. The kit of claim 25,
wherein the OX40 agonist is a monoclonal antibody that specifically binds to OX40 and comprises:
a heavy chain variable region (VH) comprising a VH complementarity determining region one (CDR1), VH CDR2, and VH CDR3 of the VH comprising the amino acid sequence shown in SEQ ID NO 7; and
a light chain variable region (VL) comprising a VL CDR1, VL CDR2, and VL CDR3 of the VL comprising the amino acid sequence shown in SEQ ID NO: 8.

27. The kit of claim 25,
wherein the 4-1BB agonist is a monoclonal antibody that specifically binds to 4-1BB and comprises:
a VH comprising a VH CDR1, VH CDR2, and VH CDR3 of the VH comprising the amino acid sequence shown in SEQ ID NO 17; and
a VL comprising a VL CDR1, VL CDR2, and VL CDR3 of the VL comprising the amino acid sequence shown in SEQ ID NO: 18.

28-44. (canceled)
